(19) United States
(12) Patent Application Publication
(10) Pub. No.: US 2006/0094065 A1
(43) Pub. Date: May 4, 2006

(54) Methods of analyzing a sample by MALDI-mass spectrometry

(76) Inventor: Viorica Lopez-Avila, Sunnyvale, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/978,261

(22) Filed: Oct. 30, 2004

(51) Int. Cl. G01N 33/53; G01N 33/00; G01N 33/558

(52) U.S. Cl. 435/7.5; 436/514; 436/86

(57) ABSTRACT

Methods of analyzing a sample by MALDI-mass spectrometry are disclosed.

### FIELD OF THE INVENTION

The invention relates generally to methods of analysis of samples using mass spectrometry.

### BACKGROUND OF THE INVENTION

Straightforward and reliable methods for simultaneously analyzing several constituents of a complex sample are extremely desirable. For example, it is desirable to determine the relative amounts of several pre-determined analytes, e.g., proteins, in blood and other bodily fluids, in medical diagnostics and other fields. However, current methodologies for sample analysis are impractical for such uses.

For example, conventional immunoassays such as ELISA, Western blots, sandwich assays and the like are typically used to assay a single pre-determined analyte, e.g., a single protein of interest. While it is possible to multiplex these assays, multiplexing is severely limited by the lack of suitable distinguishable labels. As such, conventional immunoassays if they are multiplexed, are only suitable for assaying for a very small number, e.g., two or three, analytes of interest.

Further, although immunoassays could, in theory, be performed in parallel to simultaneously analyze several analytes in a sample, parallel analysis would be impractical because the assays would require a significant amount of time, cost and effort. Performing several immunoassays in parallel also requires dividing a sample between all of the individual assays, an option that is not always available. As such, immunoassays are not practical for simultaneous analysis of several analytes in a sample.

Another current methodology that, so far, has been unsuitable for the simultaneous analysis of several analytes in a complex sample is mass spectrometry. Many biological samples are complex in that they contain tens of thousands, if not millions, of analytes. Typical mass spectrometers are unable to resolve all of the analytes of such samples because a signal from an analyte of interest may be masked by a signal from another analyte, making it impossible to assess the presence of the analyte of interest with any accuracy. Mass spectrometers, alone, are inherently unsuitable for the analysis of complex samples since they cannot adequately distinguish between the analytes of the complex samples. As a result of this, mass spectrometers are usually used in conjunction with other analyte separation devices such as gas chromatography or HPLC devices in order to separate analytes prior to their analysis in the mass spectrometer. Combining other analyte separation methods with mass spectrometry solves many of the inherent problems of mass spectrometry, but, because analyte separation equipment cannot systematically separate analytes of interest (which may be analytes having diverse biochemical or physical properties such as known components of any biochemical or signal transduction pathway) away from those that are not of interest, the mass spectrometer, so far, has found little use in simultaneous analysis of several analytes in a sample.

Accordingly, while there is a great need for methods for simultaneously analyzing several constituents of a complex sample, conventional methodologies fail to meet this need. The present invention combines novel affinity-based analyte separation methods with mass spectrometry and meets this need, and others.

References of interest include: published US Patent Applications 20010019829, 20010014461, 20020137106, 20020142343, 20020150927, 20020155509, 20020177242, 20020182649, 20020195555, 20030077616, 20030096224, 20030219731 and 20030027216; U.S. Pat. Nos. 6,630,358, 6,365,418 6,569,383, 6,309,605 and 6,197,599; and Chemova et al, Oncogene 2001, 20:5378-5392; Gubitz et al, J. Biol. Chem. 277:5631-5636; Katoh et al, Int. J. Oncol. 2003 22:1155-9; and Neubert et al, Anal. Chem. (2002) 74:3677-3683.

### SUMMARY OF THE INVENTION

The invention addresses the aforementioned deficiencies in the art, and provides novel methods for analyzing a sample which includes an analyte. In a method in accordance with the invention, the sample is contacted with a capture agent in solution. The capture agent is capable of reversibly and specifically binding to the analyte, to provide an analyte/capture agent complex. The capture agent is adapted to bind to an anchor group. The analyte/capture agent complex is contacted with a porous matrix having the anchor group bound thereto to provide an analyte/matrix complex. Next the analyte/matrix complex is washed, and a digest reagent is added to result in a solution containing digested analyte. The solution containing the digested analyte is then analyzed using MALDI-mass spectrometry.

In certain embodiments, the analyte may be eluted from the analyte/matrix complex and recovered in an eluant-fraction before the digest reagent is added to digest the analyte. In other embodiments, the analyte remains bound to the porous matrix via the capture agent during the washing to provide a washed analyte/matrix complex which is then contacted with the digest reagent to result in a solution containing digested analyte.

In some embodiments, contacting the sample with the capture agent in solution, contacting the analyte/capture agent complex with a porous matrix, washing, and adding a digest reagent are all performed in the same well of a multi-well plate, and wherein the washing includes subjecting an exit end of said well to low atmospheric pressure.

Additional objects, advantages, and novel features of this invention shall be set forth in part in the descriptions and examples that follow and in part will become apparent to those skilled in the art upon examination of the following specifications or may be learned by the practice of the invention. The objects and advantages of the invention may be realized and attained by means of the instruments, combinations, compositions and methods particularly pointed out in the appended claims.

### DETAILED DESCRIPTION

Before the invention is described in detail, it is to be understood that unless otherwise indicated this invention is not limited to particular materials, reagents, reaction materials, manufacturing processes, or the like, as such may vary. It is also to be understood that the terminology used herein is for purposes of describing particular embodiments only, and is not intended to be limiting. It is also possible in the present invention that steps may be executed in different sequence where this is logically possible. However, the sequence described below is preferred.

It must be noted that, as used in the specification and the appended claims, the singular forms “a,” “an” and “the” include plural referents unless the context clearly dictates otherwise. Thus, for example, reference to “a solid support” includes a plurality of solid supports. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

In this specification and in the claims that follow, reference will be made to a number of terms that shall be defined to have the following meanings unless a contrary intention is apparent.

The term “sample” as used herein relates to a material or mixture of materials, typically, although not necessarily, in fluid form, e.g., aqueous, containing one or more components of interest. Samples may be derived from a variety of sources such as from food stuffs, environmental materials, a biological sample such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components).

“Bound” may be used herein to indicate direct or indirect attachment. “Bound” (or “bonded”) may refer to the existence of a chemical bond directly joining two moieties or indirectly joining two moieties (e.g. via a linking group or other entity). The chemical bond may be a covalent bond, an ionic bond, a coordination complex, hydrogen bonding, van der Waals interactions, hydrophobic stacking, or non-covalent bond, or may exhibit characteristics of multiple types of chemical bonds. In certain instances, “bound” includes embodiments where the attachment is direct and also embodiments where the attachment is indirect. Depending on the context, “connected”, “linked”, or other like term indicates that two groups are bound to each other, wherein the attachment may be direct or indirect, wherein the attachment may be reversible or irreversible. “Immobilized” references a group that is bound to another moiety, e.g. covalently, non-covalently, reversibly, non-reversibly. Reversible binding indicates binding that typically may be released, e.g. by changing the conditions to promote release of a bound species. An example of reversible binding is binding of an analyte to a capture agent followed by elution of the analyte by changing the conditions under which the contact is occurring, e.g. changing pH, temperature, ionic strength, salt concentration Irreversible binding indicates that the bound species remains bound during normal performance of the methods of the present invention.

The term “analyte” is used herein to refer to a known or unknown component of a sample. The analyte may be specifically bound to another component in solution or on a support, e.g. to a capture agent on a substrate surface. In particular embodiments, the analyte (or a portion of the analyte) and the capture agent are members of a specific binding partner pair. In general, analytes are biopolymers, i.e., an oligomer or polymer such as an oligonucleotide, a peptide, a polypeptide, an antibody, or the like. In this case, an “analyte” is referenced as a moiety in a mobile phase (e.g., fluid), to be detected by a “capture agent” which, in some embodiments, is bound to a substrate, or in other embodiments, is in solution. However, either of the “analyte” or “capture agent” may be the one which is to be evaluated by the other (thus, either one could be an unknown mixture of analytes, e.g., polypeptides, to be evaluated by binding with the other). In particular embodiments, the capture agent may be labeled with a label which has an observable characteristic, providing for detection of the analyte or capture agent by detecting the detectable characteristic.

The term “capture agent” refers to an agent that binds an analyte through an interaction that is sufficient to permit the agent to bind and concentrate the analyte from a homogeneous mixture of different analytes. The binding interaction may be mediated by an affinity region of the capture agent. Representative capture agents include polypeptides and polynucleotides, for example antibodies, peptides or fragments of double stranded DNA may employed. Accordingly, the term “capture agent” refers to a molecule or a multi-molecular complex which can specifically bind an analyte, e.g. specifically bind an analyte for the capture agent with a dissociation constant (KD) of less than about 10-6 M (e.g. 10-7 M) without binding to other targets.

Capture agents usually “specifically bind” one or more analytes. The term “specific binding” refers to the ability of a capture agent to preferentially bind to a particular analyte that is present in a homogeneous mixture of different analytes. In certain embodiments, a specific binding interaction will discriminate between desirable and undesirable analytes in a sample, in some embodiments more than about 10 to 100-fold or more (e.g., more than about 1000- or 10,000-fold). In certain embodiments, the affinity between a capture agent and analyte when they are specifically bound in a capture agent/analyte complex is characterized by a KD (dissociation constant) of less than about 10-6 M (e.g. less than about 10-7 M, less than about 10-8 M, less than about 10-9 M) and typically greater than about 10-10 M.

The term “capture agent/analyte complex” is a complex that results from the specific binding of a capture agent with an analyte, i.e., a “binding partner pair”. A capture agent and an analyte for the capture agent specifically bind to each other under “conditions suitable for specific binding”, where such conditions are those conditions (in terms of salt concentration, pH, detergent, protein concentration, temperature, etc.) which allow for binding to occur between capture agents and analytes to bind in solution. Such conditions, particularly with respect to antibodies and their antigens, are well known in the art (see, e.g., Harlow and Lane (Antibodies: A Laboratory Manual Cold Spring Harbor Laboratory, Cold Spring Harbor, N.Y. (1989)). Conditions suitable for specific binding typically permit capture agents and target pairs that have a dissociation constant (KD) of less than about 10-6 M to bind to each other, but not with other capture agents or targets.

As used herein, “binding partner pairs” and equivalents refer to pairs of molecules that can be found in a capture agent/analyte complex, i.e. exhibit specific binding with each other.

The phrase “surface-bound capture agent” refers to a capture agent that is immobilized on a surface of a solid support, where the support can have a variety of configurations, e.g., a sheet, bead, or other structure, such as a plate with wells. In certain embodiments, the collections of capture agents employed herein are present on a surface of the same support, e.g., in the form of an array. In particular embodiments of the invention, the capture agent (e.g. SmB polypeptide, or an antibody) may be bound to a surface, e.g. of a solid support. In such embodiments, the capture agent may be bound directly to the surface or indirectly via an anchor group, e.g. via a linker or a binding partner. For example, in an embodiment in which an anchor group is bound to the surface (directly or indirectly), a capture agent adapted to bind to the anchor group may then bind to the anchor group and thus become indirectly bound to the surface via the anchor group.

The term “predetermined” refers to an element whose identity is known prior to its use. For example, a “pre-determined analyte” is an analyte whose identity is known prior to any binding to a capture agent. An element may be known by name, sequence, molecular weight, its function, or any other attribute or identifier. In some embodiments, the term “analyte of interest”, i.e. a known analyte that is of interest, is used synonymously with the term “pre-determined analyte”.

The term “mixture”, as used herein, refers to a combination of elements, e.g., capture agents or analytes, that are interspersed and not in any particular order. A mixture is homogeneous and not spatially separable into its different constituents. Examples of mixtures of elements include a number of different elements that are dissolved in the same aqueous solution, or a number of different elements attached to a solid support at random or in no particular order in which the different elements are not specially distinct. In other words, a mixture is not addressable. To be specific, an array of capture agents, as is commonly known in the art and described below, is not a mixture of capture agents because the species of capture agents are spatially distinct and the array is addressable.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is not found naturally. Put in other terms, “purified” or “isolated” references a substance from which a substantial portion of the sample in which the substance resides has been separated from the substance, leaving a greater proportion of the purified substance than prior to the separation.

The term “assessing” includes any form of measurement, and includes determining if an element is present or not. The terms “determining”, “measuring”, “evaluating”, “assessing” and “assaying” are used interchangeably and may include quantitative and/or qualitative determinations. Assessing may be relative or absolute. “Assessing the presence of” includes determining the amount of something present, and/or determining whether it is present or absent.

The terms “antibody” and “immunoglobulin” are used interchangeably herein to refer to a capture agent that has at least an epitope binding domain of an antibody. These terms are well understood by those in the field, and refer to a protein containing one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (IgG1, IgG2, IgG3, IgG4), delta, epsilon and mu heavy chains or equivalents in other species. Full-length immunoglobulin “light chains” (of about 25 kDa or about 214 amino acids) comprise a variable region of about 110 amino acids at the NH2-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding partner pairs, e.g., biotin (member of biotin-avidin specific binding partner pair), and the like. The antibodies may also be bound to a solid support, including, but not limited to, polystyrene plates or beads, and the like. Also encompassed by the terms are Fab′, Fv, F(ab′)2, and or other antibody fragments that retain specific binding to antigen.

Antibodies may exist in a variety of other forms including, for example, Fv, Fab, and (Fab′)2, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986)). Monoclonal antibodies and “phage display” antibodies are well known in the art and encompassed by the term “antibodies”.

A “biopolymer” is a polymer of one or more types of repeating units, regardless of the source. Biopolymers may be found in biological systems and particularly include polypeptides and polynucleotides, as well as such compounds containing amino acids, nucleotides, or analogs thereof. The term “polynucleotide” refers to a polymer of nucleotides, or analogs thereof, of any length, including oligonucleotides that range from 10-100 nucleotides in length and polynucleotides of greater than 100 nucleotides in length. The term “polypeptide” refers to a polymer of amino acids of any length, and encompasses “peptide,” which references a polymer of amino acids in the range from 6-50 amino acids in length. In general, polypeptides may be of any length, e.g., greater than 2 amino acids, greater than 4 amino acids, greater than about 10 amino acids, greater than about 20 amino acids, greater than about 50 amino acids, greater than about 100 amino acids, greater than about 300 amino acids, usually up to about 500 or 1000 or more amino acids. “Peptides” are generally greater than 2 amino acids, greater than 4 amino acids, greater than about 10 amino acids, greater than about 20 amino acids, usually up to about 50 amino acids. In some embodiments, peptides are between 5 and 30 amino acids in length.

The terms “polypeptide” and “protein” are used interchangeably. The term “polypeptide” includes polypeptides in which the conventional backbone has been replaced with non-naturally occurring or synthetic backbones, and peptides in which one or more of the conventional amino acids have been replaced with one or more non-naturally occurring or synthetic amino acids. The term “fusion protein” or grammatical equivalents thereof references a protein composed of a plurality of polypeptide components, that while not attached in their native state, are joined by their respective amino and carboxyl termini through a peptide linkage to form a single continuous polypeptide. Fusion proteins may be a combination of two, three or even four or more different proteins. The term polypeptide includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, and the like.

The term “array” encompasses the term “microarray” and refers to an ordered array of capture agents for binding to aqueous analytes and the like. An “array,” includes any two-dimensional or substantially two-dimensional (as well as a three-dimensional) arrangement of spatially addressable regions (i.e., “features”) containing capture agents, particularly antibodies, and the like. Where the arrays are arrays of proteinaceous capture agents, the capture agents may be adsorbed, physisorbed, chemisorbed, or covalently attached to the arrays at any point or points along the amino acid chain. In some embodiments, the capture agents are not bound to the array, but are present in a solution that is deposited into or on features of the array.

Any given substrate may carry one, two, four or more arrays disposed on a surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of the same or different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm.

Arrays can be fabricated using drop deposition from pulsejets of either precursor units (such as nucleotide or amino acid monomers) in the case of in situ fabrication, or the previously obtained capture agent.

An array is “addressable” when it has multiple regions of different moieties (e.g., different capture agent) such that a region (i.e., a “feature” or “spot” of the array) at a particular predetermined location (i.e., an “address”) on the array will detect a particular sequence. Array features are typically, but need not be, separated by intervening spaces.

The subject array may be an array of features, each feature corresponding to a “fluid-retaining structure”, e.g., a well, wall, hydrophobic barrier, or the like. Such arrays are well known in the art, and include 24-well, 48-well, 96-well, 192-well, 384-well and 1536-well microtiter plates, or multiple thereof. In certain embodiments, the features are delineated by a hydrophobic chemical boundary, and, accordingly, the array substrate may be planar and contain features containing a hydrophobic boundary. Features may be delineated by drawing lines between them with a hydrophobic pen (e.g., a PAP PEN from Newcomer Supply, Middleton, Wis.), for example. Other fluid retaining structures are well known in the art and include physical and chemical barriers. On one embodiment, the fluid retaining structure is formed by a bead of hydrophobic material, e.g., a bead of a viscose silicone material, around a fluid-retaining area. Capture agents may be present in the fluid retaining structure, but not necessarily bound to the surface of the array substrate.

An “array layout” refers to one or more characteristics of the features, such as feature positioning on the substrate, one or more feature dimensions, and an indication of a moiety at a given location.

The term “MALDI sample plate” refers to a device that is removably insertable into a MALDI ion source for a mass spectrometer and contains a substrate having a surface for presenting analytes for ionization by a laser. As will be described in greater below, a MALDI sample plate may contain a plurality of features, i.e., discrete, addressable regions, each containing a different analyte for ionization by the laser of the MALDI mass spectrometer.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

Accordingly, the present invention provides novel methods for analyzing a sample which includes an analyte. In a method in accordance with the invention, the sample is contacted with a capture agent in solution. In a typical embodiment, both the analyte and the capture agent are in solution when the sample is contacted with the capture agent. The capture agent is capable of reversibly and specifically binding to the analyte, to provide an analyte/capture agent complex. The capture agent is adapted to bind to an anchor group. The analyte/capture agent complex is contacted with a porous matrix having the anchor group bound thereto under conditions sufficient to allow the anchor group to bind to the analyte/capture agent complex, to provide an analyte/matrix complex. The analyte/matrix complex has the analyte bound indirectly to the matrix via the capture agent and the anchor group. Next the analyte/matrix complex is washed, and a digest reagent is added to result in a solution containing digested analyte. The solution containing the digested analyte is then analyzed using MALDI-mass spectrometry.

In certain embodiments, the analyte may be eluted from the analyte/matrix complex and recovered in an eluant-fraction before the digest reagent is added to digest the analyte. In other embodiments, the analyte remains bound to the porous matrix via the capture agent during the washing to provide a washed analyte/matrix complex which is then contacted with the digest reagent to result in a solution containing digested analyte.

In some embodiments, contacting the sample with the capture agent in solution, contacting the analyte/capture agent complex with a porous matrix, washing, and adding a digest reagent are all performed in the same well of a multi-well plate, and wherein the washing includes subjecting an exit end of said well to low atmospheric pressu

The digest reagent may be any agent that cuts the analyte to result in the solution containing the digested analyte. Agents capable of cutting proteins are known in the art and include chemical cleavage reagents and enzymatic cleavage reagents, such as trypsin, chymotrypsin, pepsin, elastase, or any other endopeptidase, in particular, an endopeptidase which cuts at a specific site. For example, trypsin will typically cut a protein at an accessible arginine or lysine residue. In particular embodiments, the digested analyte is analyzed using time-of-flight mass spectrometry, e.g. employing methods such as MALDI-TOF MS (matrix assisted desorption/ionization—time of flight mass spectrometry).

In certain embodiments of the invention, the capture agents are proteinaceous capture agents, methods for the making of which are generally well known in the art. For example, polypeptides may be produced in bacterial, insect or mammalian cells (see, e.g. Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.) using recombinant means, isolated, and deposited onto a suitable substrate.

Capture agents may be selected based on their binding to predetermined analytes in a sample. Accordingly, in the subject methods, the pre-determined analytes and the capture agents that bind those analytes are selected prior to starting the subject methods. In other embodiments, the capture agents are not pre-determined and their binding specificity may be unknown.

Capture agents may be chosen using any means possible. For example, sets of capture agents present on an array may bind to proteins of a particular signal transduction, developmental or biochemical pathway, proteins having similar biological functions, proteins of similar size or structure, or they may bind proteins that are known markers for a biological condition or disease. Capture agents may also be chosen at random, or on the availability of capture agents, e.g., if a capture agent is available for purchase, for example. In some embodiments, a capture agent may be chosen purely because it is desirable to know whether a known or unknown binding partner for that capture agent is present in a sample. The binding partner for a capture agent does not have to be known for the capture agent to be present on an array for use in the subject methods.

In particular embodiments, the capture agent is contacted with a sample in aqueous solution, and then attached via the anchor group (covalently or non-covalently) to a porous matrix (i.e., a porous insoluble solid support). Porous matrices suitable for use in the instant methods are known in the art and include, but are not limited to, beads (e.g., magnetic or paramagnetic beads, polystyrene beads, and the like); membranes; and matrices such as agarose, sepharose and the like. Well-known porous matrices include glass fibers, polystyrene, polypropylene, polyethylene, dextran, nylon, amyloses, natural and modified celluloses, polyacrylamides, agaroses, and magnetite, and the like. In particular embodiments matrices used in immunochromatography, e.g. agarose, SEPHAROSE™-brand chromatography medium, etc., may be used. See, Scopes, 1984, Protein Purification: Principles and Practice, Springer-Verlag New York, Inc., NY, Livingstone, 1974, Methods Enzymology: Immunoaffinity Chromatography of Proteins 34:723-731).

Attachment of a capture agent to a porous matrix is facilitated by using a porous matrix that is coated with an anchor group that binds to the capture agent. For example, a porous matrix may be coated with an antibody-binding agent such as protein A or protein G, or any other agent, e.g., streptavidin, avidin, glutathione, maltose, etc., that can bind a suitable capture agent, e.g., a biotinylated capture agent or a capture agent containing a GST, His-tag or MPB moiety. Binding capture agents to porous matrices using a variety of cross-linkers is also well known in the art, and is described in great detail on pages 319-330 of Harlow and Lane (Using Antibodies: A Laboratory Manual, CSHL Press, 1999). The binding of capture agents to porous matrices provide an affinity substrate (e.g., an immunoaffinity substrate), which substrates are known in the art.

In certain embodiments, capture agents may be present in the wells of a multi-well plate, e.g., a 96-well or 384-well microtitre plate, although any solid substrate, planar or with fluid-retaining structures, may be used. In particular embodiments, a well of a multi-well plate may have an entrance end through which liquid may be placed into the well, and an exit end through which liquid may be removed (i.e., may exit) from the well. If certain porous matrices are used, e.g., a slurry of beads or resin or the like, a filter element may also be present under the porous matrix to prevent the porous matrix from exiting the well through the exit end of the well. In certain embodiments, the filter element may be permeable to liquid (e.g., sample, wash or the like) but impermeable to the porous matrix. In certain other embodiments, the porous matrix used may be sufficiently robust so that a filter element is not required. In general, multi-well plates containing wells through which liquids may pass are well known in the art and may be termed “filter plates” or “filtration plates” by some manufacturers. Suitable multi-well plates containing wells through which liquids may pass that may be employed in the subject methods (e.g., filter plates) may be purchased from Pall Inc. (East Hills, N.Y.), Millipore (Billerica, Mass.), Nalge Nunc International (Rochester, N.Y.). Filter plates employable in the subject methods are described in U.S. Pat. No. 6,309,605.

A well of a multi-well filter plate, if employed in the subject methods, may contain a filter having a pore size that is smaller than size of matrix used (e.g., smaller than the diameter of the beads used). Exemplary filters may be hydrophilic or hydrophobic, or charged or uncharged, and may be made using a suitable membrane, e.g., PVDF membrane (e.g., DURAPORE™ or IMMOBILON™ PVDF membrane) or glass fiber, cellulose ester, phosphocellulose, DEAE etched polycarbonate or the like. The pore size of such filters may range in size from about 0.1 to about 10 microns (e.g., maybe about 0.22, about 0.45, about 0.65, or about 1.2 microns), although filters containing pore sizes outside of this range are readily employed in the subject methods.

In certain embodiments, a multi-well plate employed in the subject methods may be adapted for use with a vacuum pump or other means for generating a motive force that induces liquid present in a well of the plate through a filter and out of the exit end of the well. Accordingly, in certain embodiments, a plate may be adapted for connection to a manifold to which a vacuum pump may be operably connected. The manifold, when connected to an operating vacuum pump, provides a lower than atmospheric pressure at the exit end of the wells in a plate connected to the manifold and forces liquid in the wells through the filter and out of the plate. Systems for applying lower than atmospheric pressure to the wells of a multi-well plate are known in the art (e.g., the MULTISCREEN™ vacuum manifold by Millipore of Billerica, Mass. and BIOVAC96™ Vacuum Manifold by Bioneer of Rockville, Md.), and need not be described here in any greater detail than that set forth above.

In certain embodiments, the anchor group may itself be a capture agent, a specific binding partner, a reactive chemical group that is adapted to react with a complementary active group to forma bond, e.g. a covalent bond.

In other embodiments, after capture agent/analyte complexes are formed, the analytes bound in capture agent/analyte complexes are separated, e.g., eluted, from the capture agents to become free in solution. This is usually done by incubating the capture agent/analyte complexes under conditions suitable for separation of capture agent and analyte of a capture agent/analyte complex. Such conditions vary depending on the type of capture agent used and how it may be bound to a solid support, and generally involve incubating the complexes in an elution buffer that has high pH (e.g., pH 11-13), low pH (e.g., pH 1-4), high salt (e.g., 5M LiCl or 3.5 M MgCl2), ionic detergents (e.g., 1% SDS), dissociating agents (e.g., urea or guanidine HCl), chaotropic agents (e.g., thiocyanate), organic solvents (e.g., dioxane) or water, for a period of time. Elution methods for immunoaffinity protocols are very well known in the art and generally described on pages 335-339 of Harlow and Lane, supra.

After elution, the eluted analytes may be transferred directly to a MALDI sample plate, or, in other embodiments, the eluted analytes may be transferred to another substrate and prepared for MALDI analysis and then transferred to a MALDI sample plate. In these embodiments, the array may be re-used for the assessment of another sample.

In certain embodiments, in preparation for analysis by MALDI mass spectrometry, the subject analytes are cleaved, i.e., fragmented using a digest reagent, e.g., a chemical reagent, enzyme, or energy input, to result in at least one analyte fragment. A fragment can result from a sequence-specific or sequence independent cleavage event. Examples of reagents commonly used for cleaving polypeptides include enzymes, for example, proteases, such as thrombin, trypsin, chymotrypsin and the like, and chemicals, such as cyanogen bromide, acid, base, and o-iodobenzoic acid. A fragment can also be generated by collision induced dissociation (CID). Furthermore, a fragment can also result from multiple cleavage events such that a truncated polypeptide resulting from one cleavage event can be further truncated by additional cleavage events. In other words, an analyte may be cleaved using a combination of cleavage reagents and conditions.

Prior to their analysis, the digested analytes may be mixed with an energy absorbing molecule, i.e., a matrix, as is known in the art. The matrix may be a small organic, volatile compound with certain properties that facilitate the performance of MALDI. Accordingly, a matrix is selected based on a variety of factors such as the analyte of interest (such as type, size, and the like), etc. Examples of matrices include, but are not limited to, sinapinic acid (SA) and derivatives thereof; cinnamic acid and derivatives thereof such as alpha-cyano-4-hydroxycinnamic acid (HCCA); 2,5-dihydroxybenzoic acid (DHB); 3-hydroxypicolinic acid (HPA); 2′,4′,6′-trihydroxyacetophenone; and dithranol. The matrix may be dissolved in a suitable solvent that is selected at least in part so that it is miscible with the analyte solution. For example, in the analysis of peptides/proteins HCCA and SA work best with ACN/0.1%TFA as solvent and in the analysis of oligonucleotides HPA and ACN/H2O may be employed. After the matrix and analyte (which may be derivatized and/or fragmented) are mixed, the analyte/matrix mixture is transferred, i.e., spotted to a feature of a MALDI sample plate, and dried to form crystals.

The practice of the present invention will employ, unless otherwise indicated, conventional techniques of synthetic organic chemistry, biochemistry, molecular biology, and the like, which are within the skill of the art. Such techniques are explained fully in the literature.

While the foregoing embodiments of the invention have been set forth in considerable detail for the purpose of making a complete disclosure of the invention, it will be apparent to those of skill in the art that numerous changes may be made in such details without departing from the spirit and the principles of the invention. Accordingly, the invention should be limited only by the following claims.

All patents, patent applications, and publications mentioned herein are hereby incorporated by reference in their entireties.

1. A method of analyzing a sample comprising an analyte, the method comprising:
a) contacting the sample with a capture agent in solution, the capture agent capable of reversibly and specifically binding to the analyte, to provide an analyte/capture agent complex, wherein the capture agent is adapted to bind to an anchor group;
b) contacting the analyte/capture agent complex with a porous matrix having the anchor group bound thereto, to result in an analyte/matrix complex;
c) washing the analyte/matrix complex to provide a result selected from the group consisting of i) an eluant-fraction which includes the analyte, and ii) a washed analyte/matrix complex;
d) contacting the result of c) with a digest reagent to result in a solution containing digested analyte; and
e) analyzing the solution using a MALDI-MS method.

2. The method of claim 1, wherein the result of c) is the eluant-fraction including the analyte.

3. The method of claim 2, wherein the method further comprises rinsing the analyte/matrix complex prior to washing the analyte/matrix complex, and wherein the washing is performed under conditions effective to release the analyte from the porous matrix to provide the eluant-fraction.

4. The method of claim 1, wherein the result of c) is the washed analyte/matrix complex, wherein the washing is performed under conditions effective to result in the analyte remaining bound to the porous matrix via the capture agent.

5. The method of claim 4, further comprising, prior to analyzing the solution, separating the solution from the porous matrix.

6. The method of claim 1, wherein contacting the sample with the capture agent in solution is performed in a well of a multi-well plate.

7. The method of claim 1, wherein contacting the analyte/capture agent complex with the porous matrix is performed in a well of a multi-well plate, and wherein the washing includes subjecting an exit end of said well to a lower than atmospheric pressure.

8. The method of claim 1, wherein contacting the sample with the capture agent in solution, contacting the analyte/capture agent complex with the porous matrix, washing, and contacting the result of c) with the digest reagent are all performed in the same well of a multi-well plate, and wherein the washing includes subjecting an exit end of said well to a lower than atmospheric pressure.

9. The method of claim 8, further comprising conducting at least one additional analysis of at least one additional sample, each additional analysis proceeding in its own respective well of the multi-well plate.

10. The method of claim 1, wherein said porous matrix comprises beads.

11. The method of claim 1, wherein the capture agent is a polypeptide.

12. The method of claim 1, wherein the capture agent is an antibody.

13. The method of claim 1, wherein the digest reagent is a chemical or enzymatic digest reagent.

14. The method of claim 13, wherein the enzymatic digest reagent is an enzyme.

15. The method of claim 1, wherein the capture agent is non-covalently or covalently linked to the porous matrix via the anchor group.

16. The method of claim 1, wherein the anchor group is selected from biotin, an avidin, an antibody, an antibody fragment, and a reactive group adapted to covalently bind to a complementary active group attached to the capture agent.

17. The method of claim 1, wherein analyzing the solution provides results, and wherein said results are compared to results obtained from a control sample.

18. The method of claim 17, wherein the control sample does not contain any analyte that binds to said capture agent.

19. The method of claim 17, wherein the control sample comprises a known quantity of analyte that binds to said capture agent.

20. The method of claim 19, wherein said MALDI-MS method includes employing time-of-flight mass spectrometry.
